SEQUENCE LISTING (21 sequences)

SEQ ID NO: 1 - DNA, 21 bp, Artificial Sequence
  lacZ siRNA targeting sequence
gcggctgccggaatttacctt

SEQ ID NO: 2 - DNA, 21 bp, Artificial Sequence
  Ret siRNA targeting sequence
ccgctggtggactgtaataat

SEQ ID NO: 3 - DNA, 21 bp, Artificial Sequence
  Ret forward primer
ctgtgcagtcagcaagagacg

SEQ ID NO: 4 - DNA, 21 bp, Artificial Sequence
  Ret reverse primer
agcagttgcaggtgccatagc

SEQ ID NO: 5 - DNA, 20 bp, Artificial Sequence
  beta-actin forward primer
gactacctcatgaagatcct

SEQ ID NO: 6 - DNA, 20 bp, Artificial Sequence
  beta-actin reverse primer
gcggatgtccacgtcacact

SEQ ID NO: 7 - DNA, 21 bp, Artificial Sequence
  GFRa1 forward primer
agaccatcgtgcctgtgtgct

SEQ ID NO: 8 - DNA, 21 bp, Artificial Sequence
  GFRa1 reverse primer
aggtcgttcccactgttgctg

SEQ ID NO: 9 - DNA, 20 bp, Artificial Sequence
  GDNF forward primer
tgcttcctagaagagagcgg

SEQ ID NO: 10 - DNA, 21 bp, Artificial Sequence
  GDNF reverse primer
tgccctactttggtcactcac

SEQ ID NO: 11 - DNA, 20 bp, Artificial Sequence
  GAPDH forward primer
ctgcaccaccaactgcttag

SEQ ID NO: 12 - DNA, 20 bp, Artificial Sequence
  GAPDH reverse primer
aggtccaccactgacacgtt

SEQ ID NO: 13 - DNA, 19 bp, Artificial Sequence
  Ret51 forward primer
gagccctcccttccacatg

SEQ ID NO: 14 - DNA, 21 bp, Artificial Sequence
  Ret51 reverse primer
ggactctctccaggccagttc

SEQ ID NO: 15 - DNA, 23 bp, Artificial Sequence
  Ret 51 MGB probe
attgaaaacaaactctatggcat

SEQ ID NO: 16 - DNA, 22 bp, Artificial Sequence
  Ret9 forward primer
ccgctggtggactgtaataatg

SEQ ID NO: 17 - DNA, 25 bp, Artificial Sequence
  Ret9 reverse primer
gtaaatgcatgggaaattctaccat

SEQ ID NO: 18 - DNA, 14 bp, Artificial Sequence
  Ret9 MGB probe
cccctccctcgagc

SEQ ID NO: 19 - DNA, 21 bp, Artificial Sequence
  siRNA targeting RET
ccgctggtggactgtaataat

SEQ ID NO: 20 - DNA, 21 bp, Artificial Sequence
  siRNA targeting RET
taggctggttctcaaccggaa

SEQ ID NO: 21 - DNA, 23 bp, Artificial Sequence
  siRNA targeting LacZ
aagcggctgccggaatttacctt

(19) United States
(12) Patent Application Publication
(10) Pub. No.: US 2011/0195072 A1
(43) Pub. Date: Aug. 11, 2011

(54) NON-NEUROENDOCRINE CANCER THERAPY

(76) Inventors: Anne Boulay, Blotzheim (FR); Madlaina Breuleux, Basel (CH); Nancy Hynes, Basel (CH); Heidi A. Lane, Biel-Benken (CH)

(21) Appl. No.: 12/440,077

(22) PCT Filed: Sep. 10, 2007
(86) PCT No.: PCT/EP2007/007874

§ 371 Date: Mar. 5, 2009

(30) Foreign Application Priority Data
Sep. 12, 2006 (EP) 06120532.4

(51) Int. Cl. A61K 39/395; C12Q 1/68; G01N 33/53; A61K 38/00; A61K 31/7088; A61K 31/497; C07K 14/00; C07K 16/40; C07H 21/04

(52) U.S. Cl. 424/158.1; 435/6.14; 435/7.1; 514/19.3; 514/44 A; 514/253.09; 530/300; 530/387.1; 530/391.3; 536/24.5

(57) ABSTRACT

The present invention provides a method of selecting subjects suffering or being predisposed to suffering from a proliferative disease in non-neuroendocrine tissues for treatment with a Ret inhibitor. The present invention also provides Ret inhibitors as medicaments for subjects suffering or being predisposed to suffering from a proliferative disease in non-neuroendocrine tissues.

### FIELD OF THE INVENTION

The present invention relates to the expression and function of Ret receptor tyrosine kinase signalling in non-neuroendocrine tumors, in particular breast tumours, and the use of Ret inhibitors to treat proliferative disease in particular tumours of non-neuroendocrine origin presenting Ret expression.

### BACKGROUND TO THE INVENTION

Ret is the receptor tyrosine kinase for the glial-derived neurotrophic factor (GDNF) family of growth factors, that is essential for the development of the sympathetic, parasympathetic and enteric nervous system and the kidney. Ret is a paradigm of a single gene that causes different types of human cancers when targeted by different genetic alterations. Different germ line gain of function point mutations in Ret cause three related dominantly inherited cancer syndrome affecting neuroendocrine tissues: multiple endocrine neoplasia type 2A (MEN2A) and type 2B (MEN2B), and familial medullary thyroid carcinoma (FMTC). Moreover, sporadic or radiation-induced papillary thyroid carcinomas (PTC) are characterized by Ret activation following rearrangements of the tyrosine kinase domain with various partners (the fusion is referred to as Ret/PTC), resulting in ligand-independent dimerization and constitutive activation of the chimeric proteins. However there is a need to investigate the expression and role of Ret in non-neuroendocrine tumours.

The present inventors have found that Ret is expressed at the mRNA and protein level in a set of breast tumour cell lines and breast tumour biopsies. The sequence of human Ret (mRNA [coding sequence; 3218 nucleotides] and protein [1072 amino acids]) is available under GenBank accession numbers NM_{—}020630 or NM_{—}065681. In particular it has been found that Ret signalling following stimulation with the specific ligand GDNF enhances anchorage-independent proliferation of breast tumour cells. Hence Ret is a potential target for therapeutic intervention for non-neuroendocrine tumours in particular breast tumours.

### SUMMARY OF THE INVENTION

Accordingly, the present invention is based on the determination of the presence of Ret RNA and protein expression in cells which are prone to abnormal proliferation.

In a first aspect, the present invention provides a method of selecting subjects suffering or being predisposed to suffering from a proliferative disease (neoplasm) in non-neuroendocrine tissues for treatment with a Ret inhibitor and selecting those subjects displaying Ret expression and/or activity for said treatment, comprising the steps of:

providing a sample from a patient suffering or being predisposed to suffering from said disease;

detecting any Ret expression and/or activity in said sample; and

selecting those patients for treatment with a Ret inhibitor whose sample displays an increased level of Ret expression and/or activity.

Identifying a patient suffering or being predisposed to suffering from proliferative disease in non-neuroendocrine tissues can be carried out by many techniques known to the skilled reader, including immunological techniques (e.g. western blotting, ELISA, immunoprecipitation), histochemical, immunocytochemical, and immunohistochemical means, microscopy and/or gene/protein/phosphoprotein expression techniques. Someone who is predisposed to developing such disease, may not yet be fully committed to developing said disease, or may be committed, but not yet display all symptoms of the disease, such as tumour growth and/or metastasis. Nevertheless, such patients may benefit from treatment with a Ret inhibitor, in order to prevent or minimise disease progression.

The sample may be isolated from tumour tissue or a sample of tissue suspected of developing a tumour, or from a sample of body fluid, such as blood, mucous, urine or sputum. It may also be desirable to lyse cells within the sample, so as to release any Ret present. Lysis may be achieved by the use of solvents, such as SDS, sonication, mechanical disruption, a sudden drop in osmotic pressure and/or the use of one or more proteases to release some or all of any Ret present within the cells.

Optionally, it may be desirable to compare a level of Ret expression and/or activity with a control or “normal” sample, in order to detect whether or not a level of Ret expression and/or activity in the sample is increased or decreased in comparison to a control or normal sample.

A control sample is understood to be a sample from a “normal” non diseased tissue and can therefore be considered as having a “normal” level of Ret expression and/or activity. Thus, the level found in a particular tissue from a subject, e.g. a sample of tumour tissue, may be compared with a control sample, e.g. a sample of normal tissue from a subject not suffering from the disease, or a sample of normal (i.e non-tumour) tissue from the same subject. For example, increased expression and/or activity of Ret may be predictive of a beneficial therapeutic effect (i.e. an antiproliferative/cell killing effect) of a Ret inhibitor alone or in combination with, for example, a targeted or cytotoxic agent.

It is to be understood, that not all patients suffering or predisposed to suffering from said disease will display a level of Ret expression and/or activity, and as such patients would likely not be suitable for treatment with a Ret inhibitor and are not therefore encompassed by the present invention.

As well as selecting patients for treatment with a Ret inhibitor, the present invention may extend to testing such selected patients during Ret inhibitor therapy, in order to observe if Ret levels or activity decrease in response to Ret inhibitor therapy. It may also be appropriate to test patients who are undergoing other forms of therapy, such as chemotherapy and/or radiotherapy, as their Ret levels may alter, such as increase in response to such therapy and they may subsequently become appropriate for Ret inhibition therapy.

According to the method of the present invention, subjects suffering from such a proliferative disease can be screened in order to determine the expression and/or activity of Ret. The method may be performed in vitro, e.g. on a sample of tumour tissue derived from the subject.

The presence of Ret and/or its activity may be assayed in the tumour sample by any technical means on the basis of e.g. RNA expression using for example the technique of RT-PCR or on the basis of e.g. protein expression/modifications (e.g. phosphorylation) using for example the technique of Western blotting, immunocytochemistry, immunohistochemistry or immunoassays including ELISA, immunoprecipitation and electrophoresis assays.

For example, ELISA (enzyme linked immunosorbent assay) type assays, immunoprecipitation type assays, conventional Western blotting assays, immunocytochemistry and immunohistochemistry assays using e.g. monoclonal or polyclonal antibodies are also utilized to determine levels of Ret protein and activity (e.g. phosphorylation).

An example of a typical immunoassay would comprise the step of exposing a sample, as defined hereinabove, of an individual suspected or identified as having cancer, to an antibody recognizing Ret. This antibody is either a polyclonal antibody which may be raised against purified Ret protein or phosphorylated Ret (or peptide sequences derived from the Ret protein sequence) according to techniques well known in the art (cf “Antibodies. A Laboratory Manual”, Harlow et al., 1988, Cold Spring Harbor Laboratory, NY-UA) or a monoclonal antibody which may be raised against purified Ret protein or phosphorylated Ret (or Ret-derived peptides) or an immunogen preparation containing Ret and selected for its specificity and/or high affinity for Ret according to conventional techniques.

As will be recognized by those in the art, numerous types of immunoassays are available for use in the present invention. For instance homogeneous and heterogeneous assays, direct and indirect binding assays, competitive assays, sandwich assays well known in the art and described in numerous publications, e.g. “Antibodies. A Laboratory Manual”, Harlow et al., 1988, Cold Spring Harbor Laboratory, NY-USA.

The antibody recognizing Ret protein or active (e.g. phosphorylated) Ret, or another component of the test kit may carry a label depending upon their application. A “label” means here a molecule which provides, directly or indirectly, a detectable signal.

Various labels may be employed, such as radionucleotides (e.g. ¹²⁵I, ¹³¹I, technetium, indium, ³H and ¹⁴C), fluorescents, chemiluminescents, enzymes (e.g. peroxidase, alkaline phosphatase, β-D-galactosidase, glucose oxidase, glutamate decarboxylase and β-amylase), enzyme substrates, cofactors and inhibitors, particles (e.g. colloidal gold particles), combinations of ligands and receptors (e.g. streptavidin and biotin) and the like. Enzymatic labels are advantageous because they allow a high sensitivity, comparable to that of radioactive labels, provide superior spatial resolution in a histological context, do not require particular safety precautions and can be used in commercially available automated systems. Enzymatic labels most widely used both in research as diagnostic applications are horseradish peroxidase and alkaline phosphatase

Particularly useful immunoassays are sandwich assays and competitive inhibition immunoassays which use at least one monoclonal antibody as defined above.

The sandwich assay may be a homosandwich assay, a heterosandwich assay or a lectin-immunometric assay.

Polyclonal and monoclonal antibodies specific to Ret protein/post-translational modifications may be produced in accordance with known immunization methods or are commercially available (e.g. Santa Cruz Biotechnology Inc catalogue sc-167, Novocastra catalogue NCL-RET).

Ret expression may also be measured by two-dimensional (2-D) gel electrophoresis. 2-D gel electrophoresis is known in the art and typically involves isoelectric focusing (IEF) along a first dimension followed by SDS-PAGE (sodium dodecyl sulphate-polyacrylamide gel electrophoresis) along a second dimension. The resulting electropherograms are analyzed, for example, by immunoblot analysis using antibodies.

The present invention thus provides a method of screening subjects suffering from or predisposed to suffering from a proliferative disease (or neoplasm) of non-neuroendocrine origin in order to predict their responsiveness to a treatment with a Ret inhibitor, comprising detecting the presence of Ret and/or Ret activity in a sample, by a method as defined above.

The term “Ret inhibitor” as used herein includes, but is not limited, to small molecule inhibitors binding to Ret, such as by targeting the active site in the kinase domain of Ret, (see below and Mologeni et al (Protein Expression and purification, 41, (2005), 177-185)); an RNAi molecule designed to inhibit Ret expression; an antibody capable of specifically binding to Ret, such as to its kinase or extracellular domain, and interfering with Ret activity and/or binding to other proteins; neutralising aptamers against Ret, such as described in Cerchia et al (PloS Biol, 2005, 3(4):e123); or a peptide fragment of Ret or peptide mimetic which is capable of disrupting or competitively inhibiting Ret function.

One particular class of potential Ret inhibitors is disclosed in WO2006/034833 which describes a number of cyclic diaryl ureas as having tyrosine kinase inhibition activity. A preferred class is based on formula VIA disclosed therein and a particularly preferred compound for use as a Ret inhibitor is [6-[[1-[[4-[(4-methyl-1-piperazinyl)methyl]-3-(trifluoromethyl)phenylamino]carbonyl]-1H-indol-5-yl]oxy]-4-pyrimidinyl]acetamide, as described in example 43 and shown below (compound 1).

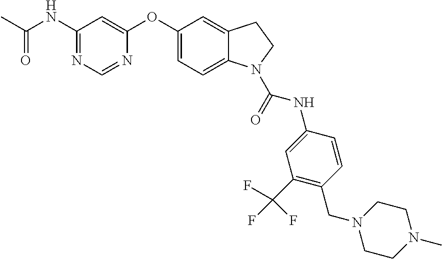

The present inventors have observed that such a compound(s) has particular utility in reducing breast cancer cell proliferation/survival and may therefore have utility in treating cancers of non-neuroendocrine origin.

Thus, in a further aspect, there is provided use of a Ret inhibitor in the manufacture of a medicament for treating a proliferative disease in non-neuroendocrine tissues, characterised by displaying Ret expression and/or activity. In certain embodiments, the Ret inhibitor may be used to treat subjects displaying an increased level of expression and/or activity in comparison to a control sample.

As described above, the patient may be suffering from, or be predisposed to developing said disease and as such the term treating can be understood to extend to prophylactic treatment as well as conventional treatment.

Preferred Ret inhibitors have been described hereinabove and in particular with respect of the preferred compounds disclosed in WO2006/034833.

In each case where citations of patent applications or scientific publications are given, the subject-matter relating to the compounds is hereby incorporated into the present application by reference. Comprised are likewise the pharmaceutically acceptable salts thereof, the corresponding racemates, diastereoisomers, enantiomers, tautomers as well as the corresponding crystal modifications of above disclosed compounds where present, e.g. solvates, hydrates and polymorphs, which are disclosed therein. The compounds used as active ingredients in the combinations of the invention can be prepared and administered as described in the cited documents, respectively.

The proliferative disease may be a benign or malignant proliferative disease of non-neuroendrocrine origin, e.g. benign prostatic hyperplasia, or a neoplastic disease, preferably a malignant proliferative disease, e.g. a cancer, e.g. tumours and/or metastasis (where ever located), e.g. brain and other central nervous system tumours (e.g. tumours of the meninges, brain, spinal cord, cranial nerves and other parts of central nervous system, e.g. glioblastomas); head and/or neck cancer; breast tumours; circulatory system tumours (e.g. heart, mediastinum and pleura, and other intrathoracic organs, vascular tumours and tumour-associated vascular tissue); excretory system tumours (e.g. kidney, renal pelvis, ureter, bladder, other and unspecified urinary organs); gastrointestinal tract tumours (e.g. oesophagus, stomach, small intestine, colon, colorectal, rectosigmoid junction, rectum, anus and anal canal), tumours involving the liver and intrahepatic bile ducts, gall bladder, other and unspecified parts of biliary tract, other and digestive organs); head and neck; oral cavity (lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumours (e.g. vulva, vagina, Cervix uteri, Corpus uteri, uterus, ovary, and other sites associated with female genital organs, placenta, penis, testis, and other sites associated with male genital organs); respiratory tract tumours (e.g. nasal cavity and middle ear, accessory sinuses, larynx, trachea, bronchus and lung, e.g. small cell lung cancer or non-small cell lung cancer); skeletal system tumours (e.g. bone and articular cartilage of limbs, bone articular cartilage and other sites); skin tumours (e.g. malignant melanoma of the skin, non-melanoma skin cancer, basal cell carcinoma of skin, squamous cell carcinoma of skin, mesothelioma, Kaposi's sarcoma); and tumours involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites, tumours of blood and lymphatic system (e.g. Hodgkin's disease, Non-Hodgkin's lymphoma, Burkitt's lymphoma, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia.

Where hereinbefore and subsequently a tumour, a tumour disease, a carcinoma or a cancer is mentioned, also metastasis in the original organ or tissue and/or in any other location are implied alternatively or in addition, whatever the location of the tumor and/or metastasis is but of non-neuroendocrine origin.

Preferably the malignancy is breast cancer.

In a further aspect, the present invention provides a method of treating a proliferative disease characterized by Ret expression and/or activity, in a subject in need thereof, comprising administering to said subject a Ret inhibitor in an amount effective to reduce and/or inhibit undesirable cell proliferation.

The Ret expression and/or activity may be increased with respect to a control sample.

The present invention in certain embodiments further provides:
i. Detection of Ret expression levels to determine the sensitivity or response of a proliferative disease of non-neuroendocrine origin in a subject to treatment with a Ret inhibitor;
ii. a method of selecting subjects suffering from a proliferative disease for treatment with a Ret inhibitor, comprising determining the expression of Ret in the tumour of the subject by a method as described above, and selecting those subjects showing Ret expression for further therapy.

The invention also concerns an imaging agent for Ret expressing cancer, such as Ret expressing breast cancer i.e. an agent for visualizing in vivo breast cancer cells, said imaging agent comprising a monoclonal antibody as defined above linked to a detectable label such as a radionuclide, e.g. ¹²⁵I, ¹³¹I, technetium or indium. Such an imaging agent would be useful for detecting tumours in situ by a method including the steps of identifying person suspected of having a tumour, introducing the imaging agent into the tissue effected, such as breast tissue and detecting (e.g., by radioimaging, using scintigraphy) the presence of the detectable label bound to said tissue, a high level of such label bound to a given site being indicative of a tumour at that site. Using such an imaging method permits a non-invasive determination of the presence, location or absence of Ret expressing tumour in a person, which would be particularly useful for monitoring the condition of a patient being treated for a tumour known to express Ret.

In another aspect the invention relates to an immunotoxin comprising a monoclonal antibody as defined above conjugated to a toxin molecule. Such conjugation may be accomplished by known chemical methodology, or, if the toxin is a protein, by means of genetically engineering a hybrid DNA molecule encoding both the toxin and Ret-binding portion of the antibody as a single polypeptide: expression of this recombinant DNA molecule would result in an immunotoxin in which the antibody portion is linked to the toxin portion by a peptide bond. Examples of naturally-occurring proteinaceous toxins that could be incorporated into the immunotoxin of the invention include diphtheria toxin, Pseudomonas exotoxin A, ricin and other plant toxins such as abrin, modeccin, volkensin, and viscumin, cholera toxin (produced by Vibrio cholerae bacteria), Shiga toxin (produced by various strains of Shigella bacteria), the so-called “Shiga-like” toxins (produced by E. coli and other enteric bacteria), Salmonella heat-labile enterotoxin and E. coli heat-labile enterotoxin. Non-proteinaceous toxins include known cytotoxic anticancer agents such as doxorubicin, as well as α-emitting radionuclides such as astatine and β-emitting nuclides such as yttrium. This immunotoxin is useful for targeting and killing tumour cells which express Ret on their surfaces.

### DETAILED DESCRIPTION

The present invention will now be further described by way of non-limiting example and with reference to the figures, which show:

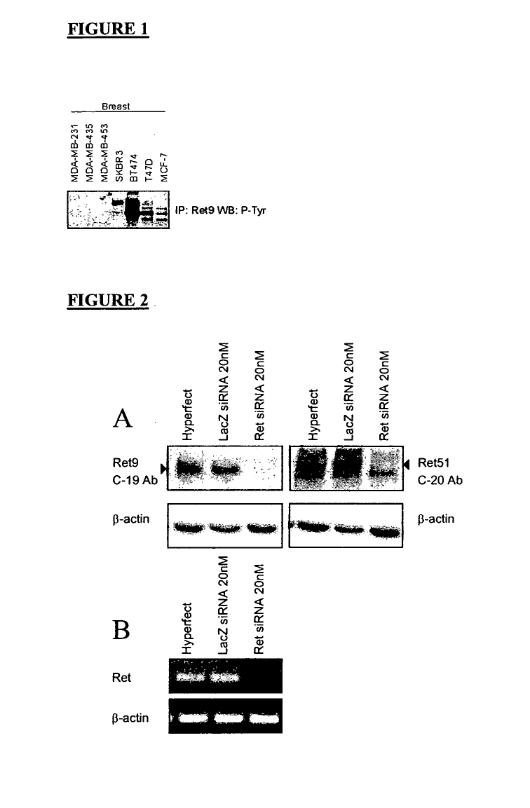

FIG. 1 shows an immunoblot of tyrosine phosphorylation analysis after immunoprecipitation of Ret 9 from a number of breast tumour cell lines;

FIGS. 2A and B show respectively a Western blot and PCR analysis of Ret expression with and without Ret siRNA treatment;

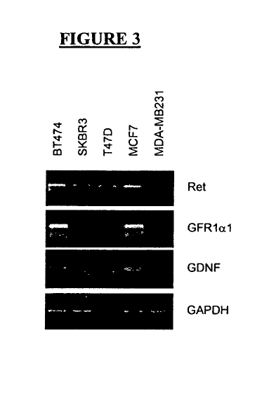

FIG. 3 shows the RNA expression by gel electrophoresis of Ret in certain breast tumour cell lines and also expression, or lack thereof, of the co-receptor GFRα1 and the ligand GDNF;

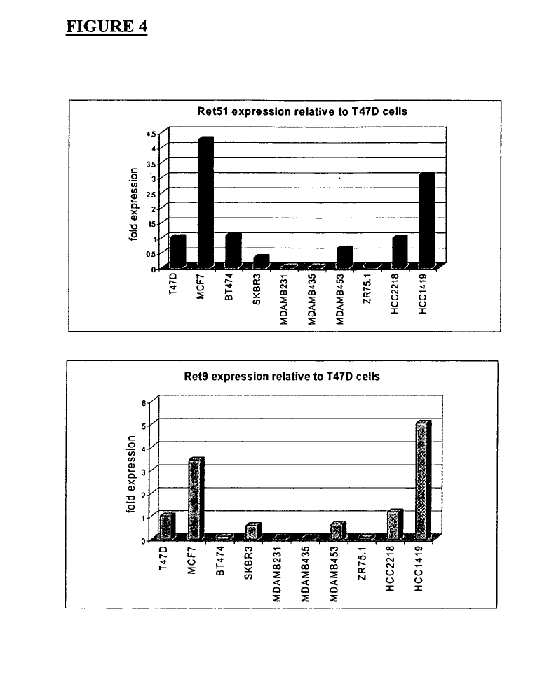

FIGS. 4A and B show a comparison of mRNA expression levels of Ret51 and Ret9 in various breast cancer cell lines, in comparison to T47D cells;

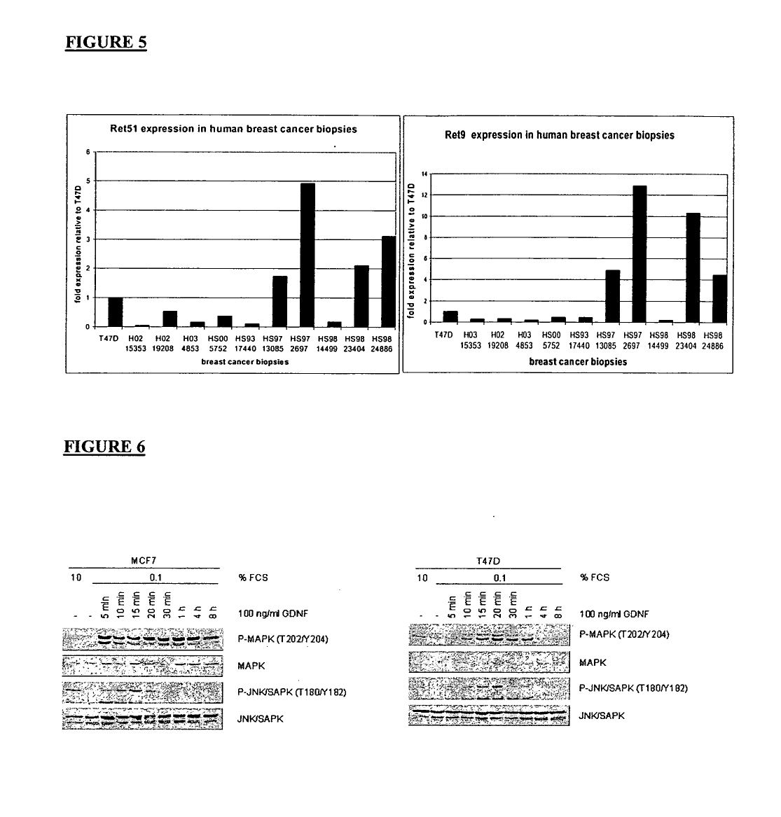

FIGS. 5A and B show mRNA expression levels of Ret51 and Ret9 from a panel of human breast cancer biopsies, in comparison to T47D cells;

FIGS. 6A and B show immunoblots of the effect of increasing concentrations of GDNF on MAPK (also known and referred to herein as Erk) and JNK/SAPK phosphorylation (activation) in MCF7 and T47D cells;

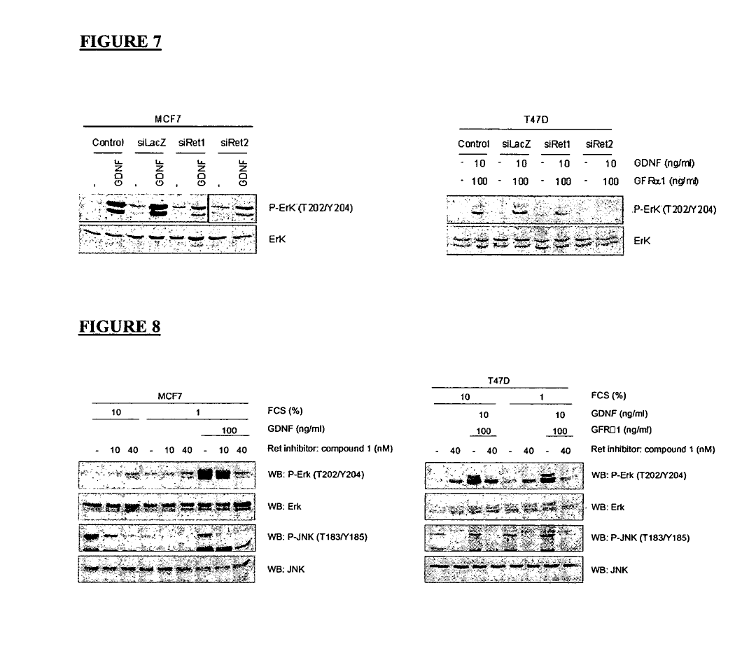

FIGS. 7A and B are immunoblots showing that transfection of siRNAs targeting Ret prevents GDNF-induced phosphorylation (activation) of Erk (also known as MAPK);

FIGS. 8A and B are immunoblots showing that GDNF enhancement of Erk (also known as MAPK) and JNK phosphorylation (activity) in breast tumour cells can be prevented by pre-treatment with a Ret inhibitor;

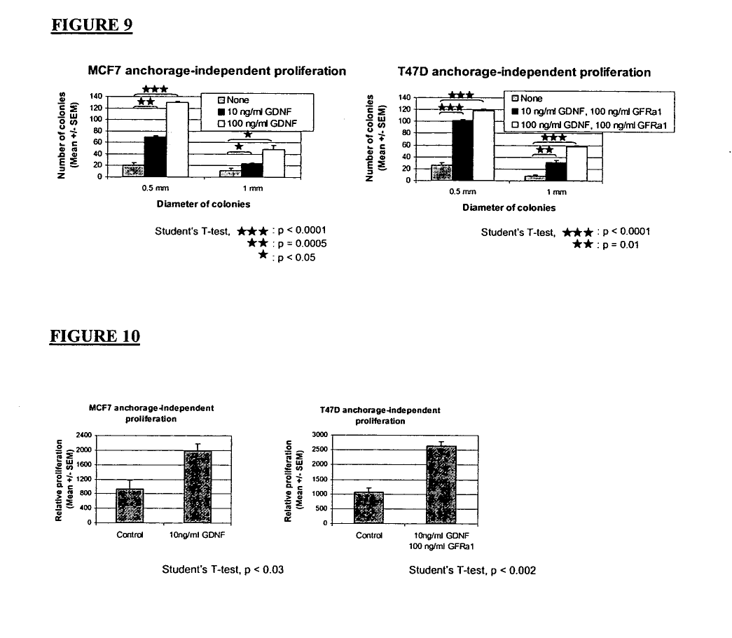

FIGS. 9A and 9B are graphs showing that GDNF stimulation enhances anchorage-independent proliferation of breast tumour cells (soft agar assays);

FIGS. 10A and B are graphs showing that GDNF stimulation significantly enhances MCF7 and T47D anchorage-independent cell proliferation (polyheme assay); and

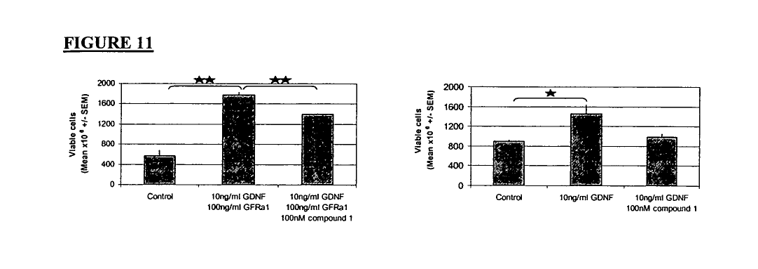

FIGS. 11A and B are graphs showing that GDNF enhancement of anchorage independent cell proliferation, can be prevented by inhibition of Ret by a Ret inhibitor (polyheme assay).

### EXAMPLE 1

The human breast cancer cell lines MCF7 (HTB-22; American Type Culture Collection, Rockville, Md., USA), T47D (HTB-133), MDA-MB-231 (HTB-26), MDA-MB-435 (obtained from G. Orend, DKBW, Basel, Switzerland), MDA-MB-453 (HTB-131), SKBR3 (HTB-30) and BT474 (HTB-20) are seeded in 10 cm plates and incubated for 2 or 3 days at 37° C. and 5% CO₂. Cell extracts (1 mg) are immunoprecipitated with 1 μg rabbit polyclonal antibody raised against Ret (sc-167, Santa Cruz Biotechnology). Immunoprecipitated proteins are resolved by 7.5% SDS-PAGE electrophoresis and immunoblot analysis is performed using mouse monoclonal antibodies raised against phosphotyrosine residues (Cell Signalling, Beverly, Mass.). Tyrosine-phosphorylated protein bands of the appropriate size are immunoprecipitated by Ret specific antibodies in SKBR3, BT474, T47D, MDA-MB-453 and MCF7 cells, suggesting that Ret proteins are expressed and phosphorylated (e.g. activated) in some breast tumour cell lines.

### Example 2

MCF7 cells are seeded at a density of 10⁵ cells/well in 6 well plates. 24 hours after seeding, cells are transfected with control lacZ siRNA or Ret siRNA (Qiagen, 96165/96166) at a final siRNA concentration of 20 nM using HiPerFect transfection reagent (Qiagen, 301705). siRNA targeting sequences are as following:

| | |
|---|---|
| lacZ: | GCGGCTGCCGGAATTTACCTT |
| Ret: | CCGCTGGTGGACTGTAATAAT |

Ret9 and Ret51 protein expression is analyzed 72 hours post siRNA transfection using polyclonal antibodies from Santa Cruz (C-19 against Ret9: sc-167; C-20 against Ret51: sc-1290) and direct Western Blot analysis with 50 μg of total protein extract (FIG. 2 Panel A). Using both antibodies, Ret siRNA specifically results in a reduction in signal, confirming the identification of Ret protein in these samples. As a control, total RNA is extracted in a parallel experiment (RNeasy, 74104, Qiagen). First-strand cDNA is synthesized using SuperScript III First-Strand Synthesis System for RT-PCR (Invitrogen, 18080-051), using random hexamer primers. PCR analysis is performed to demonstrate the downregulation of Ret mRNA expression after siRNA transfection using the following Ret primers:

| | |
|---|---|
| Ret forward primer: | CTGTGCAGTCAGCAAGAGACG |
| Ret reverse primer: | AGCAGTTGCAGGTGCCATAGC |
| β-actin forward primer: | GACTACCTCATGAAGATCCT |
| β-actin reverse primer: | GCGGATGTCCACGTCACACT |

Only in the case of the Ret siRNA is there a reduction in Ret mRNA, further confirming the specificity of the siRNA approach used (FIG. 2 Panel B).

### Example 3

Breast tumour lines (MCF7, T47D, BT474, SKBR3 and MDA-MB-231) are seeded in 10 cm plates. At 70 to 80% confluence, total RNAs are extracted (RNeasy, 74104, Qiagen). cDNAs are synthesized by reverse transcription using the AMV reverse transcriptase (600081-51, Stratagene) and random primers (272166-0.5 kV, Pharmacia). Expression levels of the Ret receptor tyrosine kinase (40 cycles) the co-receptor GFRα1 (40 cycles) and the ligand GDNF (45 cycles) are evaluated by PCR using the following primers (GAPDH as a reference for normalization):

| | |
|---|---|
| Ret forward primer: | CTGTGCAGTCAGCAAGAGACG |
| Ret reverse primer: | AGCAGTTGCAGGTGCCATAGC |
| GFRα1 forward primer: | AGACCATCGTGCCTGTGTGCT |
| GFRα1 reverse primer: | AGGTCGTTCCCACTGTTGCTG |
| GDNF forward primer: | TGCTTCCTAGAAGAGAGCGG |
| GDNF reverse primer: | TGCCCTACTTTGGTCACTCAC |
| GAPDH forward primer: | CTGCACCACCAACTGCTTAG |
| GAPDH reverse primer: | AGGTCCACCACTGACACGTT |

Ret mRNA is expressed in MCF7, T47D, BT474 and SKBR3 tumour cell lines, but not in MDA-MB-231 cells, consistent with the analysis in example 1. Moreover, BT474 and MCF7 express the co-receptor GFRα1 and the ligand GDNF (see FIG. 3).

### Example 4

Human breast cancer cell lines, including T47D, MCF7, BT474, SKBR3, MDA-MB-231, MDA-MB-435, MDA-MB-453, ZR75.1 (CRL-1500, American Type Culture Collection, Rockville, Md., USA), HCC2218 (CRL-2343), HCC1419 (CRL-2326) are seeded in 10 cm plates and incubated for 2 days at 37° C. and 5% CO₂ to reach 60-70% confluency. Total RNA is extracted using Trizol reagent (Invitrogen, 15596-026) and RNA quality is measured with the Agilent technology. First-strand cDNA is synthesized using SuperScript III First-Strand Synthesis System for RT-PCR (Invitrogen, 18080-051), using random hexamer primers. Expression levels of the Ret receptor tyrosine kinase isoforms Ret51 and Ret9 are evaluated by quantitative real-time PCR (TaqMan, Applied Biosystems) using the following primers and FAM-coupled MGB (minor groove binding) probes from Applied Biosystems (18SrRNA as a reference for normalization):

| | |
|---|---|
| Ret51 forward primer: | GAGCCCTCCCTTCCACATG |
| Ret51 reverse primer: | GGACTCTCTCCAGGCCAGTTC |
| Ret51 MGB probe: | ATTGAAAACAAACTCTATGGCAT |
| Ret9 forward primer: | CCGCTGGTGGACTGTAATAATG |
| Ret9 reverse primer: | GTAAATGCATGGGAAATTCTACCAT |
| Ret9 MGB probe: | CCCCTCCCTCGAGC |

18SrRNA Pre-developed TaqMan Assay Reagent (Applied Biosystems, 4333760T)
   Taking T47D cells as a reference cell line, Ret mRNA is expressed in 8 out of 10 human breast cancer tumour cell lines (see FIG. 4). The 2 cell lines with no Ret mRNA expression (MDA-MB-231, MDA-MB-435) are also negative for Ret phosphoprotein expression (see Example 1).

### Example 5

Human breast cancer biopsies are obtained from Cathrin Brisken (CHUV, ISREC, Epalinges, Lausanne). Total RNA is extracted (RNeasy, #74104, Qiagen). cDNAs are synthesized by reverse transcription using the AMV reverse transcriptase (600081-51, Stratagene) and random primers (272166-0.5 kV, Pharmacia). Expression levels of the Ret receptor tyrosine kinase isoforms Ret51 and Ret9 are evaluated by quantitative real-time PCR (TaqMan, Applied Biosystems) as described above (Example 4). Taking T47D cells as a reference cell line, all the human breast tumour samples express Ret51 and Ret9 mRNA, with relatively high expression in 4 out of the 10 samples analyzed (see FIG. 5). These data indicate that the Ret tyrosine kinase is expressed in human solid tumour samples from the breast.

### Example 6

MCF7 and T47D cells are seeded at 3×10⁶/15 ml and 3×10⁶/10 ml in 15 and 10 cm plates for 2 days respectively. Cells are serum deprived (0.1% FCS) for 16 to 24 hrs or refed with normal medium. Cells are stimulated with 100 ng/ml GDNF (450-10, PeProTech) for 5, 10, 20, or 30 min 1, 4 or 8 hrs. Cell extracts are resolved by 10 or 12.5% SDS-PAGE electrophoresis and immunoblot analysis is performed using rabbit polyclonal antibodies raised against Erk (MAPK: 9102, Cell Signaling Technology) phospho-Erk (P-MAPK: Thr402/Tyr204; 9101) JNK/SAPK (9251) and phospho-JNK/phospho-SAP (Thr183/Tyr185; 9252) (see FIG. 6). In these assays, GDNF triggers increased phosphorylation of Erk and JNK/SAPK, indicating that GDNF stimulation results in activation of Erk (MAPK) and JNK/SAPK signalling pathways in MCF7 and T47D breast tumour cells.

MCF7 and T47D cells are seeded at 5×10⁵/4 ml in 6 cm plates for 24 hrs. Cells are transfected with siRNAs targeting Ret (siRet1: CCGCTGGTGGACTGTAATAAT siRet2: TAGGCTGGTTCTCAACCGGAA; Qiagen) or a control siRNA targeting LacZ (AAGCGGCTGCCGGAATTTACCTT) using Oligofectamine (12252-011, Invitrogen) and HiPerFect, respectively. After 72 hrs, cells are stimulated with 10 ng/ml GDNF alone (for 10 min in MCF7) or together with 100 ng/ml GFRα1 (for 30 min in T47D; rhGFRα1/Fc chimera 714-GR, R&D Systems), extracted and resolved by 12.5% SDS-PAGE electrophoresis and immunoblot analysis is performed using rabbit polyclonal antibodies against Erk (MAPK) and phospho-Erk (p-MAPK: see FIG. 7). Transfection of siRNAs targeting Ret prevents GDNF-induced activation of Erk (MAPK), indicating that Ret mediates GDNF-induced effects on Erk (MAPK) signaling in breast tumour cells.

MCF7 and T47D cells are seeded at 2×10⁶/10 ml or 4×10⁶/15 ml in 10 or 15 cm plates, respectively. Cells are serum deprived (1% FCS) for 16 to 24 hrs or refed with normal medium. Cells are pretreated with 10 or 40 nM of a Ret inhibitor [repeat formula name from earlier in spec] or the vehicle DMSO for 80 (MCF7) or 60 min (T47D), and then treated with 10 ng/ml GDNF alone (for 10 min in MCF7) or together with 100 ng/ml GFRα1 (for 30 min in T47D). Cell extracts are prepared and resolved by 12.5% SDS-PAGE electrophoresis and immunoblot analysis is performed using rabbit polyclonal antibodies against Erk (MAPK), phospho-Erk (p-MAPK), JNK and phospho-JNK. GDNF stimulation enhances Erk (MAPK) and JNK phosphorylation in the breast tumour cells, a phenomenon prevented by pretreatment with the Ret inhibitor, indicating that Ret mediates GDNF-induced signalling (see FIG. 8).

### Example 7

MCF7 and T47D cells are treated with 10 or 100 ng/ml GDNF alone (MCF7) or together with 100 ng/ml GFRα1 (T47D) for 10 to 20 min in suspension and subsequently seeded at 10⁵/2 ml in 6-well plates in a soft agar layer. Cells are treated again with GDNF alone or together with GFRα1 every week for 4 weeks in fresh medium. Colonies are stained with nitrotetrazolium blue and counted (Artek counter 880, Dynatech Laboratories). GDNF (in the presence of GFRα1 for T47D) stimulation significantly increased the number of colonies in a concentration-dependent manner, indicating that GDNF stimulation enhances anchorage-independent proliferation of breast tumour cells (see FIG. 9).

### Example 8

MCF7 and T47D cells are seeded at 5×10⁵/4 ml in 6 cm plates coated with polyheme (P3932, Sigma) to prevent adhesion of the cells to the substratum. Cells are treated with 10 ng/ml GDNF alone (MCF7) or together with 100 ng/ml GFRα1 (T47D) for 4 days. Cells were collected, trypsinized and counted (Vi-cell XR, Beckman Coulter). GDNF stimulation significantly enhances MCF7 and T47D anchorage-independent cell proliferation (see FIG. 10).

MCF7 and T47D cells are seeded at 10⁶/4 ml in 6 cm plates coated with polyheme. Cells are pretreated with 100 nM of a Ret inhibitor [repeat name of compound from earlier] or the vehicle DMSO for 1 hr, and then treated with 10 ng/ml GDNF alone (MCF7) or together with 100 ng/ml GFRα1 (T47D) for 4 days. Cells are collected, trypsinized and counted (Vi-cell XR, Beckman Coulter). GDNF significantly enhances MCF7 and T47D anchorage-independent cell proliferation, a phenomenon prevented by inhibition of Ret using a specific inhibitor (see FIG. 11).

1. A method of selecting subjects suffering or being predisposed to suffering from a proliferative disease (neoplasm) in non-neuroendocrine tissues for treatment with a Ret inhibitor and selecting those subjects displaying Ret expression and/or activity for said treatment, comprising the steps of:
providing a sample from a patient suffering or being predisposed to suffering from said disease;
detecting any Ret expression and/or activity in said sample; and
selecting those patients for treatment with a Ret inhibitor whose sample displays Ret expression and/or activity.

2. The method according to claim 1 further comprising the step of comparing Ret expression and/or activity with a control sample in order to detect an increase or decrease in Ret expression and/or activity in the sample.

3. The method according to claim 2 wherein the patient is selected whose sample displays an increased level of Ret expression and/or activity, in comparison to the control sample.

4. The method according to claim 3 wherein the detection of Ret expression and/or activity is carried out by immunological, histochemical, immunocytochemical, immunohistochemical, microscopy and/or gene/protein/phosphoprotein expression techniques.

5. The method according to claim 4 wherein the detection of Ret expression and/or activity is carried out using an antibody specific for Ret protein or phosphorylated Ret protein.

6. The method according to claim 5 wherein the antibody is labelled.

7. The method according to claim 6 wherein the patient to be tested is undergoing another form of therapy, such as chemotherapy and/or radiotherapy.

8. The method according to claim 7 wherein the sample is a sample of tumour tissue or tissue suspected of comprising tumour cells or cells predisposed to becoming tumour cells.

9. The method according to claim 8 wherein the sample is a sample of breast tissue.

10. Use of a Ret inhibitor in the manufacture of a medicament for treating a proliferative disease of non-neuroendocrine origin, characterised by displaying Ret expression and/or activity.

11. A method of treating a proliferative disease of non-neuroendocrine origin characterized by Ret expression and/or activity, in a subject in need thereof, comprising administering to said subject a Ret inhibitor in an amount effective to reduce and/or inhibit undesirable cell proliferation.

12. The method according to claim 10 wherein the Ret inhibitor is a small Ret binding molecule; an RNAi or siRNA molecule designed to inhibit Ret expression; an antibody capable of specifically binding to Ret or its phosphorylated form and interfering with Ret activity and/or association or binding to other proteins; neutralizing aptamers against Ret; or a peptide fragment or peptide mimetic capable of disrupting or competitively inhibiting Ret function.

13. The method according to claim 12 wherein the Ret inhibitor is a molecule according to formula VIA as disclosed in WO2006/034833.

14. The method according to claim 13 wherein the Ret inhibitor is [6-[[1-[[4-[(4-methyl-1-piperazinyl)methyl]-3-(trifluoromethyl)phenylamino]carbonyl]-1H-indol-5-yl]oxy]-4-pyrimidinyl]acetamide.

15. The method according to claim 14 wherein the non-neuroendocrine disorder is a benign or malignant proliferative disease, e.g. a cancer, e.g. tumours and/or metastasis (where ever located), e.g. brain and other central nervous system tumours (e.g. tumours of the meninges, brain, spinal cord, cranial nerves and other parts of central nervous system, e.g. glioblastomas); head and/or neck cancer; breast tumours; circulatory system tumours (e.g. heart, mediastinum and pleura, and other intrathoracic organs, vascular tumours and tumour-associated vascular tissue); excretory system tumours (e.g. kidney, renal pelvis, ureter, bladder, other and unspecified urinary organs); gastrointestinal tract tumours (e.g. oesophagus, stomach, small intestine, colon, colorectal, rectosigmoid junction, rectum, anus and anal canal), tumours involving the liver and intrahepatic bile ducts, gall bladder, other and unspecified parts of biliary tract, other and digestive organs); head and neck; oral cavity (lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumours (e.g. vulva, vagina, Cervix uteri, Corpus uteri, uterus, ovary, and other sites associated with female genital organs, placenta, penis, testis, and other sites associated with male genital organs); respiratory tract tumours (e.g. nasal cavity and middle ear, accessory sinuses, larynx, trachea, bronchus and lung, e.g. small cell lung cancer or non-small cell lung cancer); skeletal system tumours (e.g. bone and articular cartilage of limbs, bone articular cartilage and other sites); skin tumours (e.g. malignant melanoma of the skin, non-melanoma skin cancer, basal cell carcinoma of skin, squamous cell carcinoma of skin, mesothelioma. Kaposi's sarcoma); and tumours involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites, tumours of blood and lymphatic system (e.g. Hodgkin's disease, Non-Hodgkin's lymphoma, Burkitt's lymphoma, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma. T-cell lymphoma or cutaneous T-cell lymphoma). Myeloid cancer includes e.g. acute or chronic myeloid leukaemia.

16. The method according to claim 15 wherein the non-neuroendocrine disorder is breast cancer.

17. An imaging agent for detecting Ret expression comprising an antibody specific for Ret linked to a detectable label.

18. An immunotoxin for use in destroying and/or reducing non-neuroendocrine cancer cells expressing Ret comprising an antibody conjugated to a toxin molecule.
